(19) United States
(12) Patent Application Publication
(10) Pub. No.: US 2019/0125779 A1
(43) Pub. Date: May 2, 2019

(54) PHARMACEUTICAL COMPOSITIONS

(71) Applicant: University of Houston System, Houston, TX (US)

(72) Inventors: Jokubas Ziburkus, Houston, TX (US); Kiran Kumar Akula, Novato, CA (US); Ahmad Abdulla, Houston, TX (US)

(73) Assignee: University of Houston System, Houston, TX (US)

(21) Appl. No.: 15/638,921

(22) PCT Filed: Dec. 30, 2015
(86) PCT No.: PCT/US2015/067994

§ 371 Date: Jun. 30, 2017

Related U.S. Application Data
(60) Provisional application No. 62/098,131, filed on Dec. 30, 2014.

(51) Int. Cl. A61K 31/7076; A61K 31/015; A61K 31/352; A61P 25/08; A61K 36/185

(52) U.S. Cl. CPC A61K 31/7076; A61K 31/015; A61K 36/185; A61P 25/08; A61K 31/352

(57) ABSTRACT

Novel treatment methods of controlling cell hyperexcitability occurring in a neurological disease or disorder associated with epileptogenesis or cardiac disorder are disclosed herein. Novel pharmaceutical compositions comprising adenosine or adenosine agonists and at least one selected from phytocannabinoids and terpenes are disclosed.

### CROSS-REFERENCE TO RELATED APPLICATIONS

This application claims priority to International Application Number PCT/US15/67994, filed Dec. 30, 2015, and to U.S. Provisional Application No. 62/098,131, filed Dec. 30, 2014, which are hereby incorporated by reference.

### FIELD

The present disclosure relates to pharmaceutical therapies and compositions for the treatment of seizures and cardiac disorders by combining phytocannabinoids and/or terpenes with known anti-convulsant and cardiac drugs such as adenosine.

### BACKGROUND

Epilepsy is a chronic neurological disorder presenting a wide spectrum of diseases that affect approximately 50 million people worldwide.

Adenosine, as the brain's endogenous anticonvulsant, is considered to be responsible for seizure arrest and postictal refractoriness. Alterations in the adenosinergic system (adenosine and its receptors) have been referred by many previous studies indicating that deficiencies or modifications in the function of this purinergic system can contribute to epileptogenesis. Due to this emerging implication of adenosine in the managing of seizures, a new field of adenosine-based therapies has been introduced including adenosine itself, adenosine receptor agonists, antagonists, adenosine kinase, and deaminase inhibitors.

Moreover, adenosine is a ubiquitous homeostatic substance released from most cells, including neurons and glia. Once in the extracellular space, adenosine modifies cell functioning by operating G-protein-coupled receptors (GPCR; A₁, A_{2A}, A_{2B}, A₃) that can inhibit (A₁) or enhance (A₂) neuronal communication. Interactions between adenosine receptors and other G-protein-coupled receptors, ionotropic receptors and receptors for neurotrophins also occur, and this might contribute to a fine-tuning of neuronal function. Manipulations of adenosine receptors influence sleep and arousal, cognition and memory, neuronal damage and degeneration, as well as neuronal maturation.

However, in addition to adenosine, other molecules have been found to interact with these receptors. The plant Cannabis sativa produces over 421 chemical compounds, including about 80 terpeno-phenol compounds named phytocannabinoids that have not been detected in any other plant. Phytocannabinoids, including, but not limited to cannabidiol (CBD), cannabidiolic acid (CBDA), tetrahydrocannabinol (THC), tetrahydrocannabinolic acid (THCA), cannabigevarin (CBGV), cannabichromene (CBC), cannabichromenic acid (CBCA), cannabigerol (CBG), cannabigerolic acid (CBGA), and major classes of terpenes found in cannabis plants are among these molecules. For instance, CBD has been found to have an antiarrhythmic effect in ischemia/reperfusion (I/R)-induced ventricular arrhythmias, and the antiarrhythmic effect of CBD can be mediated through the activation of adenosine A₁ receptor. Additionally, it has been reported to enhance adenosine receptor A_{2A} signaling via inhibition of an adenosine transporter.

The use of such cannabinoids in combination with adenosine receptor agonists is a potential therapy for controlling hippocampal neural circuit hyperexcitability occurring in a neurological disease or disorders associated with epilepsy in a subject.

It would be advantageous to combine the use of a cannabidiol such as CBD, in combination with a non-standard anti-epileptic drug to reduce high-frequency neuronal firing and sodium dependent action potentials to enhance GABA effects.

The clinical efficacy of CBD with respect to epilepsy compound has been shown to attenuate convulsions induced in animals by various procedures and to reduce Ca²⁺ oscillations under high-excitability in cultured hippocampal neurons. The molecular basis for the antiepileptic action of CBD might involve a reduction of [Ca²⁺]ᵢ, via interaction with the mitochondrial Na²⁺/Ca²⁺-exchanger.

Another phytocannabinoid that might exert antiepileptic actions is tetrahydrocannabivarin (Δ⁹-THCV). This compound acts in a manner similar to CB₁ receptor antagonists to increase—in a GABA_{A} antagonist-sensitive manner—miniature inhibitory postsynaptic currents at interneuron—Purkinje cell synapses, and to decrease Purkinje cell spike firing in the mouse cerebellum in vitro. Collectively, such results suggest that Δ⁹-THCV acts to limit excitation via increase in GABA release, an idea that is consistent with its efficacy in an experimental model of epilepsy.

Likewise, the phytocannabinoids could be used for anti-inflammatory amelioration. There is some suggestion that CBC exerts anti-inflammatory effects and modest analgesic activity in rodents.

Likewise, CBD has been effective in well-established experimental models of analgesia (neuropathic and inflammatory pain), as well as in acute (carrageenan-induced rat paw edema) and chronic (e.g. collagen induced murine arthritis) models of inflammation in rodents.

The effect of CBD on T-cells has also been investigated. CBD exerts a generalized immunosuppressive effect through a proapoptotic mechanism involving oxidative stress-dependent activation of caspase-8. Further CBD-induced T-cell suppression might be linked to its ability to suppress the transcriptional activity of activator protein-1 and nuclear factor of activated T-cells, both of which are critical regulators of IL-2 and interferon-γ.

Psoriasis is an inflammatory disease characterized by epidermal keratinocyte hyper-proliferation. The most significant mediators involved in this disorder are those associated with a dominant Th1 cytokine profile. Δ⁹-THC, CBN, and CBD were shown to inhibit keratinocyte proliferation in the low micromolar range and in a cannabinoid receptor-independent manner.

In summary, CBD exerts anti-arthritic actions through a combination of immunosuppressive and anti-inflammatory effects.

It is possible that CBD can reverse brain damage caused by cerebral ischemia as seen in rodents. The neuroprotectant effect of CBD is different from that of Δ⁹-THC in that it is: i) cannabinoid receptor-independent, ii) long-lasting, iii) observed when the drug is administered pre- and post-ischemia, and iv) not associated with the development of tolerance.

CBD seems to reduce cerebral hemodynamic impairment, improve brain metabolic activity post-insult, and reduce brain edema and seizures associated with temporary occlusion of carotid arteries and hypoxia in rodents. These neuroprotective effects are associated with extracerebral benefits such as cardiac, hemodynamic, and ventilatory improvements. The mechanism of the cerebroprotectant effect of CBD might involve an increase in cerebral blood flow mediated by the 5-HT_{1A} receptor and/or be secondary to its cannabinoid receptor-independent anti-inflammatory action. The anti-inflammatory action of CBD is associated with inhibition of monocyte/macrophages expressing high-mobility group (a non-histone DNA-binding protein which is known to induce neurofibrillation and microglial activation in the post-ischemic brain) in the infarct area (including the striatum), and to a reduction in the number of Iba1-positive and glial fibrillary acidic protein-positive cells in the striatum.

Terpenes are the major biogenic volatile organic compounds, formed as the secondary metabolites in cannabis plants with isoprene units. Over 200 terpenoids have been identified in Cannabis and these compounds are very potent and volatile. The terpenes present in the cannabis such as limonene, β-caryophyllene, α-pinene, β-myrcene, borneol, nerolidol, eugenol, elemene, terpinyl acetate, phellandrene, fenchol, citronellol, citronellal, phytol, terpinolene, terpineol, α-humulene, β-caryophyllene oxide, α-bisabolol, geraniol, valencene, p-cymene, isopulegol, menthol, guaiol, α-humulene, 1,8-cineol, camphene, etc. are per se primarily used for their biological, antibacterial, antifungal, and antioxidant properties. In addition, terpene such as linalool was shown to have sedative, anxiolytic, and anticonvulsant in pentylenetetrazol-induced convulsions in mice. Limonene is present in citrus fruits and has sedative, anti-hyperalgesic, and anti-depressive actions in preclinical studies. The essential from lemon peel such as limonene, α-pinene, β-pinene, neral, geranial, 1,8-cineole, linalool, borneol, α-terpineol, terpinen-4-ol, and β-caryophyllene inhibited the activities of acetylcholinesterase and butyrylcholinesterase in vitro, the enzymes responsible for the symptoms of Alzheimer's disease besides the inhibition of pro-oxidant induced lipid peroxidation.

Currently there is no preparation which combines the use of adenosine or other anti-epileptic or cardiac drugs with phytocannabinoids, including, but not limited to CBD, CBDA, THC, THCA, CBGV, CBC, CBCA, CBG, CBGA, and major classes of terpenes such as limonene, β-caryophyllene, α-pinene, β-myrcene, borneol, nerolidol, eugenol, elemene, terpinyl acetate, phellandrene, fenchol, citronellol, citronellal, phytol, terpinolene, terpineol, α-humulene, β-caryophyllene oxide, α-bisabolol, geraniol, valencene, p-cymene, isopulegol, menthol, guaiol, α-humulene, 1,8-cineol, and camphene found in cannabis sativa plants. Independently, both have been shown to have anti-epileptic properties but have not been used in combination. This covers a very broad spectrum of disorders that have all been associated with seizures in any way and cardiac disorders, because adenosine is an anti-arrhythmic heart medication and phytocannabinoids can control heart rates.

### SUMMARY

An embodiment of the disclosure is a method of treating a disorder in a subject in need of treatment comprising the steps of: administering to the subject a substance selected from the group consisting of adenosine, an adenosine analog, an adenosine agonist, an adenosine transport inhibitor, modulation of adenosine levels at biophase, a serotonin receptor agonist and a combination thereof; and administering to the subject at least one selected from the group consisting of a phytocannabinoid and a terpene. In an embodiment, the disorder is selected from the group consisting seizure disorders and cardiac disorders. In an embodiment, the disorder is a seizure disorder. In an embodiment, the disorder is selected from the group consisting of hippocampal neural circuit hyperexcitability, intractable epilepsy, Dravet's syndrome, febrile seizures, autism spectrum disorder and attention deficit hyperactivity disorder. In an embodiment, the substance is selected from the group consisting of substances used to treat epilepsy, Bechet' syndrome, Dravet Syndrome, Lennox Gastaut Syndrome, intractable childhood epilepsies, Autism, Fragile x syndrome, Angelman's syndrome, multiple sclerosis, migraines, seizures in Alzheimer's disease, posttraumatic chronic pain, chronic traumatic encephalopathy, neuropathic pain, traumatic brain injury, cluster headaches, fibromyalgia, arthritis, pancreatitis, gastritis, inflammatory bowel syndrome, Crohn's disease, diabetes, gastric reflux, acid reflux syndrome, anxiety, depression, post-traumatic stress disorder, posttraumatic epilepsy, Parkinson's, glaucoma, Huntington's, and stroke. In an embodiment, the adenosine agonist is selected from the group consisting of an adenosine receptor congener, N6-cyclopentyladenosine, N6-cyclohexyladenosine, 2-chloro-cyclopentyladenosine, N-(3(R))-tetrahydrofuranyl)-6-aminopurine riboside, or a nucleoside transporter. In an embodiment, the adenosine transport inhibitor is selected from the group consisting of dipyridamole, nitrobenzylthioinosine, dilazep, benzodiazepine, dihydropyridies, xanthine or quinoline derivatives. In an embodiment, the phytocannabinoid is selected from the group consisting of CBD, CBDA, THC, THCA, CBGV, CBC, CBCA, CBG, and CBGA. In an embodiment, the terpene is selected from the group consisting of limonene, β-caryophyllene, α-pinene, β-myrcene, borneol, nerolidol, eugenol, elemene, terpinyl acetate, phellandrene, fenchol, citronellol, citronellal, phytol, terpinolene, terpineol, α-humulene, β-caryophyllene oxide, α-bisabolol, geraniol, valencene, p-cymene, isopulegol, menthol, guaiol, α-humulene, 1,8-cineol, and camphene. In an embodiment, the method further comprises administering a GABA modulating composition. In an embodiment, the GABA modulating composition is selected from the group consisting of barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien 2-yl)butanoic acid, 4-amino-3-(5-methylthien-35 2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3 amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

An embodiment of the disclosure is a pharmaceutical composition comprising: at least one substance selected from the group consisting of an adenosine, an adenosine agonist, an adenosine agonist, an adenosine transport inhibitor, a serotonin receptor agonist and a combination thereof; and at least one selected from the group consisting of a phytocannabinoid and a terpene. In an embodiment, the substance is selected from the group consisting of substances used to treat epilepsy, Bechet' syndrome, Dravet Syndrome, Lennox Gastaut Syndrome, intractable childhood epilepsies, Autism, Fragile x syndrome, Angelman's syndrome, multiple sclerosis, migraines, seizures in Alzheimer's disease, posttraumatic chronic pain, chronic traumatic encephalopathy, neuropathic pain, traumatic brain injury, cluster headaches, fibromyalgia, arthritis, pancreatitis, gastritis, inflammatory bowel syndrome, Crohn's disease, diabetes, gastric reflux, acid reflux syndrome, anxiety, depression, post-traumatic stress disorder, posttraumatic epilepsy, Parkinson's, glaucoma, Huntington's, and stroke. In an embodiment, the adenosine agonist is selected from the group consisting of an adenosine receptor congener, N6-cyclopentyladenosine, N6-cyclohexyladenosine, 2-chloro-cyclopentyladenosine, N-(3(R))-tetrahydrofuranyl)-6-aminopurine riboside, and a nucleoside transporter. In an embodiment, the adenosine transport inhibitor is selected from the group consisting of dipyridamole, nitrobenzylthioinosine, dilazep, benzodiazepine, dihydropyridies, xanthine and quinoline derivatives. In an embodiment, the phytocannabinoid is selected from the group consisting of CBD, CBDA, THC, THCA, CBGV, CBC, CBCA, CBG, and CBGA. In an embodiment, the terpene is selected from the group consisting of limonene, β-caryophyllene, α-pinene, β-myrcene, borneol, nerolidol, eugenol, elemene, terpinyl acetate, phellandrene, fenchol, citronellol, citronellal, phytol, terpinolene, terpineol, α-humulene, β-caryophyllene oxide, α-bisabolol, geraniol, valencene, p-cymene, isopulegol, menthol, guaiol, α-humulene, 1,8-cineol, and camphene. In an embodiment, the method further comprises a GABA modulating composition. In an embodiment, the GABA modulating composition is selected from the group consisting of barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien 2-yl)butanoic acid, 4-amino-3-(5-methylthien-35 2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3 amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid. In an embodiment, the pharmaceutical composition is used to treat a disorder selected from the group consisting of hippocampal neural circuit hyperexcitability, intractable epilepsy, Dravet's syndrome, febrile seizures, autism spectrum disorder and attention deficit hyperactivity disorder.

Other objects, features and advantages of the present disclosure will become apparent from the following detailed description. It should be understood, however, that the detailed description and the specific examples, while indicating embodiments of the disclosure, are given by way of illustration only, since various changes and modifications within the spirit and scope of the disclosure will become apparent to those skilled in the art from this detailed description.

### DETAILED DESCRIPTION

### Definitions

We show the particulars shown herein by way of example and for purposes of illustrative discussion of the certain embodiments of the present disclosure only. We present these particulars to provide what we believe to be the most useful and readily understood description of the principles and conceptual aspects of various embodiments of the disclosure. In this regard, we make no attempt to show structural details of the disclosure in more detail than is necessary for the fundamental understanding of the disclosure. We intend that the description should be taken with the drawings. This should make apparent to those skilled in the art how the several forms of the disclosure are embodied in practice.

We mean and intend that the following definitions and explanations are controlling in any future construction unless clearly and unambiguously modified in the following examples or when application of the meaning renders any construction meaningless or essentially meaningless. In cases where the construction of the term would render it meaningless or essentially meaningless, we intend that the definition should be taken from Webster's Dictionary 3^{rd} Edition.

As used herein, the term “mammal” is known in the art, and exemplary mammals include humans, primates, bovines, porcines, canines, felines, and rodents (e.g., mice and rats).

As used herein, the terms “patient,” “subject,” or “host” to be treated by the subject method can mean either a human or non-human mammal.

As used herein, the term “pharmaceutically-acceptable salts” is art-recognized and refers to the relatively non-toxic, inorganic, and organic acid addition salts of compounds, including, for example, those contained in compositions of the present disclosure.

As used herein, the term “prodrug” is art-recognized and is intended to encompass compounds which, under physiological conditions, are converted into the active agents of the present disclosure. A common method for making a prodrug is to select moieties which are hydrolyzed under physiological conditions to provide the desired compound. In other embodiments, the prodrug is converted by an enzymatic activity of the host animal or the target bacteria.

As used herein, the term “treating” is art-recognized and refers to curing as well as ameliorating at least one symptom of any condition or disease.

As used herein, the term “contacting” is art-recognized and refers to any method of delivering an adenosine A₁ receptor agonist, for example, but not limited to, N⁶-cyclopentyladenosine (CPA) and/or any other pharmaceutical, drug, or therapeutic compound to hippocampal or other neurological tissue. In vitro or ex vivo this can be by exposing the hippocampal or other neurological tissue to the A₁ agonist, pharmaceutical, etc. in a suitable medium, solution, or bath. In vivo any known method of administration of the A₁ agonist, pharmaceutical, etc. is suitable as described herein.

### Embodiments

The present disclosure relates to the preparation or combination of adenosine or anti-epileptic drugs with cannabinoids, including, but not limited to cannabidiol (CBD), cannabidiolic acid (CBDA), tetrahydrocannabinoid (THC), THCA, CBGV, CBC, CBCA, CBG, CBGA, and major classes of terpenes including, but not limited to limonene, β-caryophyllene, α-pinene, β-myrcene, borneol, nerolidol, eugenol, elemene, terpinyl acetate, phellandrene, fenchol, citronellol, citronellal, phytol, terpinolene, terpineol, α-humulene, β-caryophyllene oxide, α-bisabolol, geraniol, valencene, p-cymene, isopulegol, menthol, guaiol, α-humulene, 1,8-cineol, or camphene found in cannabis sativa plants.

In certain embodiments, the combinations prevent or control the occurrence or severity of epileptic seizures. In other embodiments, the combinations prevent or control cardiac disorders due the anti-arrhythmic effects. This covers a very broad spectrum of disorders that have all been associated with seizures in any way and cardiac disorders, because adenosine (adenocard) is an anti-arrhythmic heart medication, and phytocannabinoids and terpenes can control heart rates. Representative examples of a subject include but are not limited to one with intractable epilepsy, cerebral ischemia, traumatic brain injury, Dravet syndrome, febrile seizures, autism spectrum disorder, or attention deficit hyperactivity disorder.

In one embodiment of the present disclosure, seizure events are associated with hippocampal neural circuit hyper excitability occurring in a neurological disease or disorder. In this embodiment, the hippocampus of a subject in need of such therapy is contacted with a combination of compounds effective to restore excitatory/inhibitor balance thereby controlling the neural circuit hyper excitability.

In another embodiment of the present disclosure, there is provided a method of treating a neurological disease or disorder associated with epileptogenesis in a subject in need of such treatment, comprising the step of administering an amount of an adenosine A₁ agonist pharmacologically effective to block epileptogenic activities without blocking excitatory synaptic transmission. Representative examples of useful compounds include but are not limited to adenosine, an adenosine mimetic, an adenosine modulator, an adenosine transport inhibitor, and an adenosine receptor agonist.

In yet another embodiment of the present disclosure, there is provided a method of treating severe myoclonic epilepsy in a subject in need of such treatment, comprising the step of administering an amount of an adenosine A₁ agonist pharmacologically effective to treat said severe myoclonic epilepsy.

In another embodiment of the present disclosure, there is provided a method of treating a neurological disease or disorder associated with epileptogenesis in a subject in need of such treatment, comprising the step of administering an amount of an adenosine A₁ agonist pharmacologically effective to block epileptogenic activities without blocking excitatory synaptic transmission. Representative examples of useful compounds include but are not limited to adenosine, an adenosine mimetic, an adenosine modulator, an adenosine transport inhibitor, and an adenosine receptor agonist.

In yet another embodiment of the present disclosure, there is provided a method of treating severe myoclonic epilepsy in a subject in need of such treatment, comprising the step of administering an amount of an adenosine A₁ agonist pharmacologically effective to treat said severe myoclonic epilepsy.

Representative examples of useful compounds include but are not limited to adenosine, an adenosine mimetic, an adenosine modulator, an adenosine transport inhibitor, and an adenosine receptor agonist.

Representative examples of phytocannabinoids that activate the adenosine A₁ receptor, enhance adenosine receptor A_{2A} signaling via inhibition of an adenosine transporter, or a combination thereof include but are not limited to adenosine transport inhibitor dipyridamole, nitrobenzylthioinosine, dilazep, benzodiazepine, dihydropyridies, xanthine, or quinoline derivatives, and major classes of terpenes found in cannabis plants are among these molecules.

Representative examples of adenosine receptor agonists include but are not limited to an adenosine receptor congener, N⁶-cyclopentyladenosine, N⁶-cyclohexyladenosine, 2-chloro-cyclopentyladenosine, N-(3(R))-tetrahydrofuranyl)-6-aminopurine riboside, or a nucleoside transporter.

Representative examples of adenosine transport inhibitors include but are not limited to a dipyridamole, nitrobenzylthioinosine, dilazep, benzodiazepine, dihydropyridies, xanthine, or quinoline derivatives.

Representative examples of adenosine modulators include but are not limited to an ecto-′-nucleotidase inhibitor, an adenosine kinase inhibitor, an S-adenosylhomocysteine hydrolase inhibitor, and an adenosine diaminase inhibitor.

In certain embodiments, the pharmaceutical compositions can further comprise administrating a GABA modulating composition, an anticonvulsant agent, an ion channel inactivator, or a combination thereof. In such embodiments, the aforementioned compositions can be administered in the same formulation, in separate formulations, at the same time, or at different times than the phytocannabinoids, or various adenosine, an adenosine mimetic, an adenosine modulator, an adenosine transport inhibitor, and an adenosine receptor agonist. Likewise, pharmaceutical compositions not including a GABA modulating composition, an anticonvulsant agent, an ion channel inactivator, or a combination thereof as indicated above, can still be administered in their individual components at the same time or different times. Likewise, the components can be formulated as a composition that is administered such that all components are administered to the subject at the same time within the composition. It is to be understood that any of these pharmaceutical compositions, separate components and the like can have time release properties. In such embodiments, administration at the same time will include administration of either the pharmaceutical composition or the individual components to the subject and will not include differential time release rates.

Representative examples of a GABA modulating composition include but are not limited to barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien 2-yl)butanoic acid, 4-amino-3-(5-methylthien-35 2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3 amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

### Pharmaceutical Formulations

The dosage of any compositions of the present disclosure will vary depending on the symptoms, age, and body weight of the patient, the nature and severity of the disorder to be treated or prevented, the route of administration, and the form of the subject composition. Any of the subject formulations can be administered in a single dose or in divided doses.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible effects on the timing of administration of the formulation, can need to be identified for any particular composition of the present disclosure. The effectiveness of any subject composition and method of treatment or prevention can be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like.

While the subject is being treated, the health of the patient can be monitored by measuring one or more of the relevant indices at predetermined times during the treatment period. Treatment, including composition, amounts, times of administration and formulation, can be optimized according to the results of such monitoring. The patient can be periodically reevaluated to determine the extent of improvement by measuring the same parameters. Adjustments to the amount(s) of subject composition administered and possibly to the time of administration can be made based on these reevaluations. Treatment can be initiated with smaller dosages which are less than the optimal dose of the compound. The dosage can be increased by small increments until the optimal therapeutic effect is attained.

Agents of the present disclosure can be administered orally, parenterally, for example, subcutaneously, intravenously, intramuscularly, intraperitoneally, by intranasal instillation, or by application to mucous membranes, such as, that of the nose, throat, and bronchial tubes. They can be administered alone or with suitable pharmaceutical carriers, and can be in solid or liquid form such as, tablets, capsules, powders, solutions, suspensions, or emulsions.

The active agents of the present disclosure can be orally administered, for example, with an inert diluent, or with an assimilable edible carrier, or they can be enclosed in hard or soft shell capsules, or they can be compressed into tablets, or they can be incorporated directly with the food of the diet. For oral therapeutic administration, these active agents can be incorporated with excipients and used in the form of tablets, capsules, elixirs, suspensions, syrups, and the like. Such compositions and preparations should contain at least 0.1% of active agent. The percentage of the agent in these compositions can, of course, be varied and can conveniently be between about 2% to about 60% of the weight of the unit. The amount of active agent in such therapeutically useful compositions is such that a suitable dosage will be obtained. In certain embodiments, compositions according to the present disclosure are prepared so that an oral dosage unit contains between about 1 and 250 mg of active agent.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin.

When the dosage unit form is a capsule, it can contain, in addition to materials of the above type, a liquid carrier, such as a fatty oil. Various other materials can be present as coatings or to modify the physical form of the dosage unit. For instance, tablets can be coated with shellac, sugar, or both. A syrup can contain, in addition to the active ingredient, sucrose as a sweetening agent, methyl, and propylparabens as preservatives, a dye, and flavoring such as cherry or orange flavor.

These active agents can also be administered parenterally. Solutions or suspensions of these active agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are examples of liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents of the present disclosure can also be administered directly to the airways in the form of an aerosol. For use as aerosols, the agents of the present disclosure in solution or suspension can be packaged in a pressurized aerosol container together with suitable propellants, for example, hydrocarbon propellants like propane, butane, or isobutane with conventional adjuvants. The materials of the present disclosure also can be administered in a non-pressurized form such as in a nebulizer or atomizer.

From the foregoing description, one of ordinary skill in the art can easily ascertain the essential characteristics of this disclosure, and without departing from the spirit and scope thereof, can make various changes and modifications to adapt the disclosure to various usages and conditions. For example, we do not mean for references such as above, below, left, right, and the like to be limiting but rather as a guide for orientation of the referenced element to another element. A person of skill in the art should understand that certain of the above-described structures, functions, and operations of the above-described embodiments are not necessary to practice the present disclosure and are included in the description simply for completeness of an exemplary embodiment or embodiments. In addition, a person of skill in the art should understand that specific structures, functions, and operations set forth in the above-described referenced patents and publications can be practiced in conjunction with the present disclosure, but they are not essential to its practice.

1. A method of treating a disorder in a subject in need of treatment comprising the steps of:
administering to the subject a substance selected from the group consisting of adenosine, an adenosine analog, an adenosine agonist, an adenosine transport inhibitor, modulation of adenosine levels at biophase, a serotonin receptor agonist and a combination thereof; and
administering to the subject at least one selected from the group consisting of a phytocannabinoid and a terpene.

2. The method of claim 1 wherein the disorder is selected from the group consisting seizure disorders and cardiac disorders.

3. The method of claim 2 wherein the disorder is a seizure disorder.

4. The method of claim 1 wherein the disorder is selected from the group consisting of hippocampal neural circuit hyperexcitability, intractable epilepsy, Dravet's syndrome, febrile seizures, autism spectrum disorder and attention deficit hyperactivity disorder.

5. The method of claim 1 wherein the substance is selected from the group consisting of substances used to treat epilepsy, Bechet' syndrome, Dravet Syndrome, Lennox Gastaut Syndrome, intractable childhood epilepsies, Autism, Fragile x syndrome, Angelman's syndrome, multiple sclerosis, migraines, seizures in Alzheimer's disease, posttraumatic chronic pain, chronic traumatic encephalopathy, neuropathic pain, traumatic brain injury, cluster headaches, fibromyalgia, arthritis, pancreatitis, gastritis, inflammatory bowel syndrome, Crohn's disease, diabetes, gastric reflux, acid reflux syndrome, anxiety, depression, post-traumatic stress disorder, posttraumatic epilepsy, Parkinson's, glaucoma, Huntington's, and stroke.

6. The method of claim 1, wherein the adenosine agonist is selected from the group consisting of an adenosine receptor congener, N⁶-cyclopentyladenosine, N⁶-cyclohexyladenosine, 2-chloro-cyclopentyladenosine, N-(3(R))-tetrahydrofuranyl)-6-aminopurine riboside, or a nucleoside transporter.

7. The method of claim 1, wherein the adenosine transport inhibitor is selected from the group consisting of dipyridamole, nitrobenzylthioinosine, dilazep, benzodiazepine, dihydropyridies, xanthine or quinoline derivatives.

8. The method of claim 1, wherein the phytocannabinoid is selected from the group consisting of CBD, CBDA, THC, THCA, CBGV, CBC, CBCA, CBG, and CBGA.

9. The method of claim 1 wherein the terpene is selected from the group consisting of limonene, β-caryophyllene, α-pinene, β-myrcene, borneol, nerolidol, eugenol, elemene, terpinyl acetate, phellandrene, fenchol, citronellol, citronellal, phytol, terpinolene, terpineol, α-humulene, β-caryophyllene oxide, α-bisabolol, geraniol, valencene, p-cymene, isopulegol, menthol, guaiol, α-humulene, 1,8-cineol, and camphene.

10. The method of claim 1, further comprising administering a GABA modulating composition.

11. The method of claim 10 wherein the GABA modulating composition is selected from the group consisting of barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien 2-yl)butanoic acid, 4-amino-3-(5-methylthien-35 2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3 amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

12. A pharmaceutical composition comprising:
at least one substance selected from the group consisting of an adenosine, an adenosine agonist, an adenosine agonist, an adenosine transport inhibitor, a serotonin receptor agonist and a combination thereof; and
at least one selected from the group consisting of a phytocannabinoid and a terpene.

13. The pharmaceutical composition of claim 12, wherein the substance is selected from the group consisting of substances used to treat epilepsy, Bechet' syndrome, Dravet Syndrome, Lennox Gastaut Syndrome, intractable childhood epilepsies, Autism, Fragile x syndrome, Angelman's syndrome, multiple sclerosis, migraines, seizures in Alzheimer's disease, posttraumatic chronic pain, chronic traumatic encephalopathy, neuropathic pain, traumatic brain injury, cluster headaches, fibromyalgia, arthritis, pancreatitis, gastritis, inflammatory bowel syndrome, Crohn's disease, diabetes, gastric reflux, acid reflux syndrome, anxiety, depression, post-traumatic stress disorder, posttraumatic epilepsy, Parkinson's, glaucoma, Huntington's, and stroke.

14. The pharmaceutical composition of claim 12, wherein the adenosine agonist is selected from the group consisting of an adenosine receptor congener, N6-cyclopentyladenosine, N6-cyclohexyladenosine, 2-chloro-cyclopentyladenosine, N-(3(R))-tetrahydrofuranyl)-6-aminopurine riboside, and a nucleoside transporter.

15. The pharmaceutical composition of claim 12, wherein the adenosine transport inhibitor is selected from the group consisting of dipyridamole, nitrobenzylthioinosine, dilazep, benzodiazepine, dihydropyridies, xanthine and quinoline derivatives.

16. The pharmaceutical composition of claim 12, wherein the phytocannabinoid is selected from the group consisting of CBD, CBDA, THC, THCA, CBGV, CBC, CBCA, CBG, and CBGA.

17. The pharmaceutical composition of claim 12, wherein the terpene is selected from the group consisting of limonene, β-caryophyllene, α-pinene, β-myrcene, borneol, nerolidol, eugenol, elemene, terpinyl acetate, phellandrene, fenchol, citronellol, citronellal, phytol, terpinolene, terpineol, α-humulene, β-caryophyllene oxide, α-bisabolol, geraniol, valencene, p-cymene, isopulegol, menthol, guaiol, α-humulene, 1,8-cineol, and camphene.

18. The pharmaceutical composition of claim 12, wherein further comprising a GABA modulating composition.

19. The pharmaceutical composition of claim 12, wherein the GABA modulating composition is selected from the group consisting of barbiturates, benzodiazepines, Gabapentin, Pregabalin, 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien 2-yl)butanoic acid, 4-amino-3-(5-methylthien-35 2-yl)butanoic acid, 4-amino-3-(2-imidazolyl)butanoic acid, 4-guanidino-3-(4-chlorophenyl)butanoic acid, (3-aminopropyl)phosphonous acid, (4-aminobut-2-yl)phosphonous acid, sodium butyrate, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3 amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methylphosphinic acid, (3-aminopropyl)hydroxymethylphosphinic acid, (5-aminopent-3-yemethylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

20. The pharmaceutical composition of claim 12 wherein the pharmaceutical composition is used to treat a disorder selected from the group consisting of hippocampal neural circuit hyperexcitability, intractable epilepsy, Dravet's syndrome, febrile seizures, autism spectrum disorder and attention deficit hyperactivity disorder.
